(19) United States
(12) Patent Application Publication
(10) Pub. No.: US 2026/0308559 A1
(43) Pub. Date: Oct. 8, 2026

(54) PROPHYLACTIC/THERAPEUTIC AGENT FOR PREGNANCY-INDUCED HYPERTENSION

(71) Applicants: NATIONAL UNIVERSITY CORPORATION KANAZAWA UNIVERSITY, Ishikawa (JP); MEDICAL CORPORATION SANEIKAI, Aichi (JP); NATIONAL UNIVERSITY CORPORATION TOKAI NATIONAL HIGHER EDUCATION AND RESEARCH SYSTEM, Aichi (JP); ROHTO PHARMACEUTICAL CO., LTD., Osaka (JP)

(72) Inventors: Kunio MATSUMOTO, Ishikawa (JP); Ryu IMAMURA, Ishikawa (JP); Yukio KATO, Ishikawa (JP); Yusuke MASUO, Ishikawa (JP); Shigehiko MIZUTANI, Aichi (JP); Hiroaki KAJIYAMA, Aichi (JP); Masato YOSHIHARA, Aichi (JP)

(73) Assignees: NATIONAL UNIVERSITY CORPORATION KANAZAWA UNIVERSITY, Ishikawa (JP); MEDICAL CORPORATION SANEIKAI, Aichi (JP); NATIONAL UNIVERSITY CORPORATION TOKAI NATIONAL HIGHER EDUCATION AND RESEARCH SYSTEM, Aichi (JP); ROHTO PHARMACEUTICAL CO., LTD., Osaka (JP)

(21) Appl. No.: 19/482,960

(22) PCT Filed: May 10, 2024
(86) PCT No.: PCT/JP2024/017419

§ 371 Date: Jun. 11, 2026

(30) Foreign Application Priority Data
May 12, 2023 (JP) 2023-078958

(51) Int. Cl. C12N 9/48; A61K 38/00; A61P 9/12

(52) U.S. Cl. CPC C12N 9/485; A61P 9/12; A61K 38/00; C07K 2319/30; C12Y 304/11007

(57) ABSTRACT

The present invention addresses the problem of providing a prophylactic and/or therapeutic agent for hypertensive disorders of pregnancy, which has a physical property or a molecular weight that inhibits the passage through the placental barrier, has a pharmacological activity that mimics the original physiological function of the placenta and therefore has fewer adverse side effects, is safe for a mother body and a fetus, is stable for a long period, and can exert the efficacy thereof in vivo for a long period. The present invention is a prophylactic/therapeutic agent for pregnancy-induced hypertension, which comprises aminopeptidase A (APA) as an active ingredient, the prophylactic/therapeutic agent being characterized in that the APA is bound to an immunoglobulin Fc region through a peptide linker.

### TECHNICAL FIELD

The present invention relates to an aminopeptidase A (APA)-Fc fusion protein, and to a prophylactic and/or therapeutic agent for pregnancy-induced hypertension comprising the APA-Fc fusion protein.

### BACKGROUND ART

Hypertensive disorders of pregnancy are said to occur as many as one in five pregnant women, and are a disease that requires careful attention, because it is particularly prone to becoming severe in early-onset cases that occur before the 34th week of pregnancy, which requires particular attention. When the condition becomes severe, it can lead to elevated blood pressure, proteinuria, convulsive seizure (eclampsia), cerebral hemorrhage, and dysfunction of the liver or the kidney(s), which potentially makes the disease fatal for the mother. Furthermore, the hypertensive disorders of pregnancy can cause serious symptoms not only for the mother but also for the fetus, such as fetal growth restriction, fetal dysfunction, and premature separation of placenta, which makes it a serious disease for both the mother and the fetus.

Currently, a treatment for the hypertensive disorders of pregnancy primarily consists of rest and hospitalization. There is no method for radical cure for this condition, and it is believed that the most effective treatment is premature termination of the pregnancy. While advances in neonatal intensive care units (NICUs) in recent years have made it possible to deliver an extremely low birth weight infant with weighing less than 1,000 g, premature termination of the pregnancy means premature termination of the fetal growth. Thus, it is known that an extremely low birth weight infant or a low birth weight infant are at an increased risk of various complications involving the respiratory system, the circulatory system, the digestive system, and the nervous system, as well as developmental disorders later in life. In other words, although the premature termination of the pregnancy may protect the mother and the fetus from the serious disease, that is, the hypertensive disorders of pregnancy, it is by no means a desirable solution.

On the one hand, antihypertensive drugs are generally used as agents for lowering the blood pressure, but the “Best Practice Guide 2021 for Care and Treatment of hypertensive disorders of pregnancy” states that “there are limited evidence regarding safety and efficacy of antihypertensive drugs for mothers and fetuses.” Further, the aforementioned Practice Guide recommends the use of methyldopa, labetalol, nifedipine, and hydralazine for the hypertensive disorders of pregnancy, but each comes with notes regarding a side effect and a method of administration, and requires cautious administration. Further, these antihypertensive drugs are low molecular weight compounds with low molecular weights less than 400, and they can pass through the placental barrier and affect a fetal-placental circulatory system, which impairs the fetal-placental blood flow (leads to a reduction in a fetal blood pressure). In essence, conventional antihypertensive drugs just impose stress on the fetus and compromise fetal well-being.

Meanwhile, research on the mechanism of onset of the hypertensive disorders of pregnancy has been advanced (Non Patent Literature 1). Non Patent Literature 1 explains the mechanism of onset of the hypertensive disorders of pregnancy as follows. The fetus secretes vasopressin and angiotensin II from the pituitary gland in order to maintain its own circulatory system, and thus maintains the blood pressure. If excessive amounts of fetal vasopressin and angiotensin II are released into the maternal side, maternal blood pressure rises, leading to hypertension. An aminopeptidase A (APA), which is highly expressed in the placenta and released into the maternal blood flow, degrades angiotensin II in the placenta and the maternal blood, thereby preventing excessive transfer of the angiotensin II from the placenta (to the maternal side). As the fetal growth stimulates increased secretion of the angiotensin II, the APA secretion is also stimulated accordingly, thereby maintaining the placental barrier. The concentration of the angiotensin II, which acts as a fetal growth factor and is essential for maintaining the fetal-placental circulatory system, is always higher on the fetal side than on the maternal side. However, if an APA activity in the placental or the maternal blood decreases, undegraded angiotensin II enters into the maternal blood. It is believed that this leakage of the angiotensin II into the maternal blood raises the maternal blood pressure and triggers the hypertensive disorders of pregnancy.

Accordingly, in order to treat the hypertensive disorders of pregnancy, attempts have been made to enhance the APA activity reduced in the placenta and maternal blood by administering an APA protein (Patent Literature 1 and Non Patent Literature 2). However, in an experiment where an APA protein was administered to rats with spontaneously developed hypertension, after the administration of the APA protein, the antihypertensive effect disappeared within about three to 24 hours, and thus, daily administration of the APA protein was required. Moreover, conventional technologies (Patent Literature 1 and Non Patent Literature 2) involve production of the APA by incorporating DNA encoding the APA into a baculovirus to infect Sf-9 cells with the baculovirus. Since the Sf-9 cells are derived from insects, and their glycosylation patterns differ from those of mammalian cells, they may cause immunogenicity.

From such viewpoints, a more ideal therapeutic agent for the hypertensive disorders of pregnancy is required that it should not pass through the placental barrier, to be safe for the mother and the fetus while reliably lowering the blood pressure on the maternal side, and moreover maintain a stable and sustained effect. At present, however, no such antihypertensive drug exists.

### CITATION LIST

### Patent Literature

Patent Literature 1: JP 2007-217395 A

### Non Patent Literature

Non Patent Literature 1: Obstetrical and Gynecological Practice, 1995, Vol. 44, pp. 1819-1827
Non Patent Literature 2: Journal of Biomedicine and Biotechnology, Vol. 2011, Article ID 286947, 12 pages

### SUMMARY OF INVENTION

### Technical Problem

In light of the aforementioned circumstances, the present invention aims to provide a prophylactic and/or therapeutic agent for hypertensive disorders of pregnancy, which has physical properties or a molecular weight that prevents it from passing through the placental barrier, exhibits a pharmacological action that mimics the natural physiological function of the placenta and thus has safety for the mother and the fetus with less side effects, and is also stable over a long period and capable of exerting a sustained efficacy in the body.

### Solution to Problem

In order to solve the above problem, the present inventors diligently studied on addition of a globulin protein to a genetically engineered APA (aminopeptidase A) expressed at a high yield in a mammalian-derived cell, for the purpose of ensuring a sufficient molecular weight. As a result, the present inventors discovered an optimal globulin protein to be added to the APA for securing its pharmacological efficacy and stability. Furthermore, the present inventor found that the addition of the globulin protein to the APA via a linker having a specific sequence and a certain length, instead of directly adding the globulin protein to the APA, results in a prophylactic and/or therapeutic agent for hypertensive disorders of pregnancy which is long-lastingly stable, capable of exerting a sustained efficacy in the body, and also has excellent activity to degrade angiotensin II. Accordingly, the summary of the present invention is as follows.

[1] A prophylactic and/or therapeutic agent for pregnancy-induced hypertension, comprising an aminopeptidase A (APA) as an active ingredient, wherein the APA is bound to an immunoglobulin Fc region via a peptide linker.

[2] The prophylactic and/or therapeutic agent for pregnancy-induced hypertension according to [1], wherein the peptide linker consists of 5 to 60 amino acids.

[3] The prophylactic and/or therapeutic agent for pregnancy-induced hypertension according to [1] or [2], wherein the peptide linker comprises at least one amino acid sequence selected from the group consisting of SEQ ID NOs: 4 to 9.

[4] The prophylactic and/or therapeutic agent for pregnancy-induced hypertension according to [3], further comprising a pharmaceutically acceptable carrier.

[5] An aminopeptidase A (APA)-Fc fusion protein, wherein the aminopeptidase A (APA) binds to an immunoglobulin Fc region via a peptide linker comprising at least one amino acid sequence selected from the group consisting of SEQ ID NOs: 10 to 12.

[6] The APA-Fc fusion protein according to [5], comprising the amino acid sequence of SEQ ID NO: 11.

### Advantageous Effects of Invention

According to the present invention, it is possible to provide a prophylactic and/or therapeutic agent for hypertensive disorders of pregnancy that has physical properties and a molecular weight preventing it from crossing the placental barrier, exhibits a pharmacological action mimicking the natural physiological functions of the placenta and is therefore safe for both the mother and the fetus with fewer side effects, and is also stable over a long period and capable of exerting sustained efficacy in the body. The approach of adding a globulin protein to a recombinant protein via a linker has already been put to practical use in protein-based pharmaceuticals that bind to and act on specific receptors. However, in the case of the enzyme targeted by the present invention, differences in substrate-dependent affinity and accessibility to catalytic sites affect enzymatic activity. Therefore, the present invention differs completely in its linker introduction strategy from that of conventional protein-based pharmaceuticals. In the present invention, by evaluating enzymatic activity against a physiological substrate as the target, it is possible to provide a linker introduction strategy specifically designed for enzyme-globulin proteins.

### BRIEF DESCRIPTION OF DRAWINGS

FIG. 1 is a schematic diagram illustrating the present invention. A recombinant APA-Fc fusion protein, in which an APA that hydrolyzes angiotensin capable of vasoconstriction to increase the blood pressure, is bound to the immunoglobulin, exhibits low fetal transferability due to its large molecular size and can be used as a safe antihypertensive agent for hypertensive disorders of pregnancy.

FIG. 2 is a diagram illustrating results of measurement of a substrate hydrolytic activity of various APA-Fc fusion proteins.

FIG. 3 is a diagram illustrating results of measurement of a Glu-MCA hydrolytic activity of the various APA-Fc fusion proteins.

FIG. 4 is a graph illustrating transitions of plasma concentrations over time of the various APA-Fc fusion proteins in mice.

FIG. 5 is a schematic diagram of an evaluation experiment for antihypertensive effects of the various APA-Fc fusion proteins in induced hypertensive model mice.

FIG. 6 is a graph illustrating changes over time in systolic blood pressure after administration of the various APA-Fc fusion proteins to the induced hypertensive model mice.

FIG. 7 is a graph illustrating antihypertensive effects by administration of rhAPA-Fc in mice with hypertensive disorders of pregnancy.

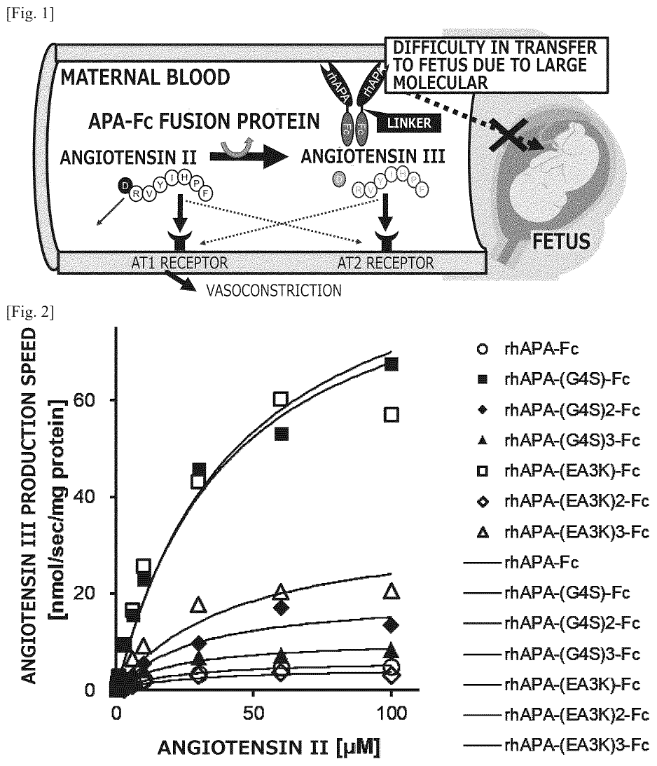

### DESCRIPTION OF EMBODIMENTS

Hereinafter, the prophylactic and/or therapeutic agent for pregnancy-induced hypertension and the APA-Fc fusion protein of the present invention will be described in detail.

### Definitions

In the present specification, the terms “peptide,” “polypeptide,” and “protein” are used synonymously and refer to polymers of amino acids. In various embodiments, the terms “peptide,” “polypeptide,” and “protein” refer to amino acid chains in which α-carbons are linked via peptide bonds. A terminal amino acid at one end of the chain (the amino terminus) has a free amino group, whereas a terminal amino acid at the other end of the chain (the carboxyl terminus) has a free carboxyl group.

In the present specification, the terms “peptide,” “polypeptide,” and “protein” also encompass peptides, polypeptides, and proteins that have been modified by any means and for any reason, including, for example, for reducing sensitivity to proteolysis, for reducing sensitivity to oxidation, for altering a binding affinity for the purpose of forming a protein complex, for altering a binding affinity, and for adding or modifying other physicochemical properties or functional properties. For example, a single amino acid substitution (for example, a conservative amino acid substitution) may be introduced, or multiple amino acid substitutions may be introduced in a natural sequence (for example, in a polypeptide portion outside a domain forming intermolecular contact). The term “conservative amino acid substitution” refers to a substitution of an amino acid in a peptide with another functionally similar amino acid. The following six groups each include amino acids that are functionally similar to each other:
(1) alanine (A), serine(S), and threonine (T);
(2) aspartic acid (D) and glutamic acid (E);
(3) asparagine (N) and glutamine (Q);
(4) arginine (R) and lysine (K);
(5) isoleucine (I), leucine (L), methionine (M), and valine (V); and
(6) phenylalanine (F), tyrosine (Y), and tryptophan (W).

In the present specification, the term “polynucleotide” is used interchangeably with “nucleic acid,” “gene,” or “nucleic acid molecule,” and refers to a polymer of nucleotides. In the present specification, the term “nucleotide sequence” is used interchangeably with “nucleic acid sequence” or “base sequence” and is represented as a sequence of deoxyribonucleotides (abbreviated as A, G, C, and T). For example, the phrase “a polynucleotide including a nucleotide sequence of SEQ ID NO: 1 or a fragment thereof” means a polynucleotide including a sequence represented by the deoxynucleotides A, G, C, and/or T of SEQ ID NO: 1, or a fragment thereof.

In the present specification, the term “Fc region” defines a C-terminal region of an immunoglobulin heavy chain and can be produced by papain digestion of an intact antibody. The Fc region may be an Fc region of a naturally occurring sequence, or may be a variant Fc region. The Fc region of the immunoglobulin typically includes two constant domains known as a CH2 domain and a CH3 domain, and may optionally include a CH4 domain.

In the present specification, the term “linker” refers to a molecule that links two molecules through a covalent bond, through an ionic bond, through a van der Waals bond, or through a hydrogen bond. For example, it also includes a nucleic acid molecule that hybridizes to a 5′ end of one complementary sequence and further hybridizes to a 3′ end of another complementary sequence, thereby linking the two non-complementary sequences.

In the present specification, the term “vector” refers to a polynucleotide that can be used to introduce another nucleic acid linked thereto into a cell. One type of vector is a “plasmid,” which refers to a linear or a circular double-stranded DNA molecule into which an additional nucleic acid segment can be linked. Other types of vectors include viral vectors (for example, replication deficient retroviruses, replication deficient adenoviruses, and replication deficient adeno-associated viruses), and additional DNA segments can be introduced into genomes of the viruses. Certain vectors are capable of autonomous replication in a host cell into which they are introduced (for example, bacterial vectors including a bacterial origin of replication and episomal mammalian vectors). Other vectors (for example, non-episomal mammalian vectors) are incorporated into a genome of a host cell after introduction into the host cell and are replicated along with the host genome. An “expression vector” is a type of vector that can direct the expression of a selected polynucleotide.

<Prophylactic and/or Therapeutic Agent for Pregnancy-Induced Hypertension>

The prophylactic and/or therapeutic agent for pregnancy-induced hypertension of the present invention (the prophylactic and/or therapeutic agent) is a prophylactic and/or therapeutic agent for pregnancy-induced hypertension, comprising an aminopeptidase A (APA) as an active ingredient, wherein the APA is bound to an immunoglobulin Fc region via a peptide linker (an APA-Fc fusion protein). In the prophylactic and/or therapeutic agent for pregnancy-induced hypertension according to the present invention, the aforementioned APA is bound to the immunoglobulin Fc region via the peptide linker. Thus, the prophylactic and/or therapeutic agent has a sufficiently large molecular weight and does not pass through the placental barrier when administered to a pregnant woman, without risks of causing adverse effects to the fetal-placental circulatory system. Furthermore, APA metabolically degrades angiotensin II, which is a vasoconstrictive peptide, into angiotensin III, and thus is capable of effectively lowering an average blood pressure. Therefore, the APA can be suitably used for the prophylactic and/or therapeutic agent for pregnancy-induced hypertension. Further, the prophylactic and/or therapeutic agent for pregnancy-induced hypertension according to the present invention comprises, as an active ingredient, the APA protein which is highly present in human blood and organs, and is produced in a mammalian cell. Therefore, even if the prophylactic and/or therapeutic agent is administered to a human subject, it is almost unlikely to produce an antibody, and therefore causes very minimal side effects on the human body.

The prophylactic and/or therapeutic agent for pregnancy-induced hypertension of the present invention may comprise, in addition to the APA-Fc fusion protein that is included as an essential ingredient (a protein with a structure in which the APA is bound to the immunoglobulin Fc region via the peptide linker), other ingredients as long as they do not impair the effects of the present invention. The APA-Fc fusion protein and other ingredients are described below.

### [APA-Fc Fusion Protein]

The APA-Fc fusion protein comprised in the prophylactic and/or therapeutic agent for pregnancy-induced hypertension of the present invention is a fusion protein having the structure (APA)-(linker)-(Fc), in which the aminopeptidase A (APA) is bound to the immunoglobulin Fc region via a peptide linker.

In the present invention, the aminopeptidase A (APA), also referred to as angiotensinase, is an enzyme that metabolically degrades the vasoconstrictive peptide angiotensin II into angiotensin III. The APA in the APA-Fc fusion protein is preferably a human APA. Further, the APA is preferably modified from its original membrane-bound form to an extracellularly secreted form by replacing a specific region at an N-terminus of a human APA with a signal peptide. In detail, it is preferable to use a modified APA in which 71 amino acids from the N-terminus of human APA are replaced with the signal peptide shown in SEQ ID NO: 1 (that is, the signal peptide of human trypsin II) (rhAPA: recombinant human APA). Such an APA modified into an extracellularly secreted form preferably has the amino acid sequence shown in SEQ ID NO: 2 or an amino acid sequence having at least 90%, 95%, or 98% sequence identity thereto.

The immunoglobulin Fc region (Fc) in the APA-Fc fusion protein of the present invention is preferably an Fc region of IgG, and may be an Fc region of any of IgG1, IgG2, IgG3, and IgG4. Further, the aforementioned Fc region preferably includes one or more amino acid deletions, additions, and/or substitutions which reduce or eliminate Fc receptor binding and/or an effector function of the Fc region. In detail, examples of the immunoglobulin Fc region (Fc) in the APA-Fc fusion protein of the present invention include, for example, the amino acid sequence of the immunoglobulin Fc region of the IgG1 shown in SEQ ID NO: 3. Here, the amino acid sequence shown in SEQ ID NO: 3 above more preferably includes a substitution of the amino acid corresponding to P329 of the Fc region of the wild-type human IgG1 (specifically, a substitution to P329G), and at least one further amino acid substitution. It further preferably includes substitutions of amino acids corresponding to P329, L234, and L235 of the Fc region of the wild-type human IgG1 (specifically, substitutions to P329G, L234A, and L235A, respectively). In SEQ ID NO: 3, the amino acids corresponding to P329, L234, and L235 of the Fc region of the wild-type human IgG1 are the 109th (P), the 14th (L), and the 15th (L), respectively, in SEQ ID NO:3. Further, the immunoglobulin Fc region (Fc) in the APA-Fc fusion protein of the present invention also includes amino acid sequences having at least 90%, 95%, or 98% sequence identity with these sequences.

The peptide linker that connects the APA and the immunoglobulin Fc region is preferably a flexible linker of a specific sequence having a certain length, and is preferably a liker consisting of 5 to 60 amino acids, more preferably a linker consisting of 5 to 30 amino acids, and even more preferably a linker consisting of 5 to 20 amino acids. In detail, examples of such linkers include GGGGS ((G4S), SEQ ID NO: 4), GGGGSGGGGS ((G4S) 2, SEQ ID NO: 5), GGGGSGGGGSGGGGS ((G4S) 3, SEQ ID NO: 6), EAAAK ((EA3K), SEQ ID NO: 7), EAAAKEAAAK ((EA3K) 2, SEQ ID NO: 8), and EAAAKEAAAKEAAAK ((EA3K) 3, SEQ ID NO: 9). Among these, linkers including GGGGS (SEQ ID NO: 4), GGGGSGGGGSGGGGS (SEQ ID NO: 6), or EAAAKEAAAKEAAAK (SEQ ID NO: 9) are preferred, and linkers including GGGGSGGGGSGGGGS ((G4S) 3, SEQ ID NO: 6) are more preferred. The preferred peptide linker is one of GGGGS ((G4S), SEQ ID NO: 4), GGGGSGGGGS ((G4S) 2, SEQ ID NO: 5), GGGGGGGGSGGGGS ((G4S) 3, SEQ ID NO: 6), EAAAK ((EA3K), SEQ ID NO: 7), EAAAKEAAAK ((EA3K) 2, SEQ ID NO: 8), or EAAAKEAAAKEAAAK ((EA3K) 3, SEQ ID NO: 9), and more preferably is GGGGS (SEQ ID NO: 4), GGGGSGGGGSGGGGS (SEQ ID NO: 6), or EAAAKEAAAKEAAAK (SEQ ID NO: 9), with GGGGSGGGGSGGGGS ((G4S) 3, SEQ ID NO: 6) being even more preferred.

In the APA-Fc fusion protein of the present invention, it is sufficient that each of the components forming the (APA)-(linker)-(Fc) structure has the aforementioned characteristics, and the protein is not limited to specific amino acid sequences. However, examples of the protein include those having the amino acid sequences represented by SEQ ID NOs: 10 to 12.

### [Method of Producing APA-Fc Fusion Protein]

In the present invention, the APA-Fc fusion protein can be produced by a method known to those skilled in the art. That is, a polynucleotide encoding the APA-Fc fusion protein in which the APA, the peptide linker, and the Fc region are fused together may be introduced into an expression vector, a suitable host cell may be transformed with the expression vector, and the APA-Fc fusion protein can be produced through purification.

The polynucleotide encoding the APA-Fc fusion protein may be functionally linked to one or multiple regulatory nucleotide sequences within the expression construct. The regulatory sequences are those approved in the field of the art and selected to promote expression of the APA-Fc fusion protein. In other words, the term “regulatory sequence” includes promoters, enhancers, and other expression regulatory elements. Typically, the regulatory sequence may include, but are not limited to, a promoter sequence, a leader sequence or a signal sequence, a ribosome binding site, a transcription start sequence and a transcription termination sequence, a translation start sequence and a translation termination sequence, and an enhancer sequence or an activator sequence. A constitutive promoter or an inducible promoter known in the art is used. The promoter may be a naturally occurring promoter, or may be a hybrid promoter composed of two or more promoter elements. The expression construct may reside inside the cell or episome, such as a plasmid, or it may be integrated into a chromosome. In various embodiments, the expression vector includes a selective marker gene to enable selection of transformed host cells. Such selective marker genes are known in the art, and may vary depending on the host cell used.

The polynucleotide encoding the APA-Fc fusion protein is provided in a state included in an expression vector that comprises a nucleotide sequence functionally linked to at least one regulatory sequence. The expression vector refers to a plasmid, a phage, a virus, or a vector for expressing a polypeptide from a polynucleotide sequence. Suitable vectors for expression in host cells are readily available, and insertion of a nucleic acid molecules into a vector is carried out using standard recombinant DNA techniques. Such vectors may include various expression regulatory sequences, which controls expression of a DNA sequence, when operably linked. Examples of such useful expression regulatory sequences include immediate early promoters of cytomegalovirus or adenovirus, early promoters and late promoters of SV40, a tet promoter, an RSV promoter, a lac system, a trp system, a TAC system, or a TRC system, a T7 promoter directed by T7 RNA polymerase, a major operator region and promoter region of 2 phage, a regulatory region for fd coat protein, a promoter for 3-phosphoglycerate kinase or other glycolytic enzymes, a promoter for acid phosphatase (for example, PhoS), a promoter of yeast a mating factor, polyhedrin promoter of baculovirus systems, and other sequences known to regulate gene expression in prokaryotic cells or eukaryotic cells or viruses thereof, as well as combinations thereof. The design of the expression vector may vary depending on factors such as the choice of host cells to be transformed and/or the type of protein desired to be expressed. Additionally, considerations may suitably be given to vector copy number, the ability to control the copy number, and expression of any other proteins (for example, antibiotic markers) encoded by the vector.

Some mammalian expression vectors include both prokaryotic sequences that promote proliferation of the vector in bacteria and one or multiple eukaryotic transcription units expressed in eukaryotic cells. Examples of the mammalian expression vector suitable for transfection into eukaryotic cells include a vector derived from pcDNAI/amp, a vector derived from pcDNAI/neo, a vector derived from pRc/CMV, a vector derived from pSV2gpt, a vector derived from pSV2neo, a vector derived from pSV2-dhfr, a vector derived from pTk2, a vector derived from pRSVneo, a vector derived from pMSG, a vector derived from pSVT7, a vector derived from pko-neo, and a vector derived from pHyg. Some of these vectors are modified by sequences derived from bacterial plasmids such as pBR322 to promote replication and drug resistance selection in both prokaryotic cells and eukaryotic cells. Alternatively, for transient expression of the protein in eukaryotic cells, derivatives of viruses such as bovine papillomavirus (BPV-1) or Epstein-Barr virus (for example, pHEBo, pREP-derived, and p205) can be used. Various methods used for preparation of the plasmid and transformation of the host cell are well known in the art. In some cases, a baculovirus expression system may be used to express a recombinant polypeptide. Examples of such baculovirus expression systems include vectors derived from pVL (for example, pVL1392, pVL1393, and pVL941), vectors derived from pAcUW (for example, pAcUW1), and vectors derived from pBlueBac (for example, pBlueBacIII-containing B-gal).

Cells for expressing and producing the APA-Fc fusion protein of the present invention are not particularly limited, but preferred examples include Expi293F cells and CHO cells. Examples of vectors for that purpose include, for instance, a Pcmv-Script vector (Stratagene, La Jolla, California), a pcDNA4 vector (Invitrogen, Carlsbad, California), and a pCI-neo vector (Promega, Madison, Wisconsin). The APA-Fc fusion protein can be expressed and produced in CHO cells proliferated in a culture medium, and then purified.

The APA-Fc fusion protein of the present invention can be purified by a protein purification method well known to those skilled in the art. Partial or complete purification (that is, purification to homogeneity) can be achieved by purification using methods such as chromatography or electrophoresis. Various methods suitable for purification are well known to those skilled in the art. Examples of such methods include, for example, precipitation using ammonium sulfate, PEG, antibodies (immunoprecipitation), or precipitation by heat denaturation followed by centrifugal separation; chromatography, such as affinity chromatography (using a protein A column), ion-exchange chromatography, gel filtration chromatography, reverse-phase chromatography, hydroxyapatite chromatography, and hydrophobic interaction chromatography; isoelectric focusing; gel electrophoresis; and combinations of these methods.

### [Other Optional Ingredients]

The prophylactic and/or therapeutic agent for pregnancy-induced hypertension of the present invention may further comprise a pharmaceutically acceptable carrier. Such a pharmaceutically acceptable carrier is well known to those skilled in the art, and may be comprised for the purpose of altering, maintaining, or preserving a pH, an osmolar concentration, a viscosity, a transparency, a color, an isotonicity, a smell, sterility, stability, a dissolution rate, a release rate, adsorption, a permeability, or the like, of the prophylactic and/or therapeutic agent for pregnancy-induced hypertension of the present invention. Preferred carriers include, but are not limited to: amino acids (for example, glycine, glutamine, asparagine, arginine, or lysine); antibacterial agents; antioxidants (for example, ascorbic acid, sodium sulfite, or sodium bisulfite); buffers (for example, borates, bicarbonates, Tris-HCl, citrates, phosphates, or other organic acids); bulking agents (for example, mannitol, or glycine); chelating agents (for example, ethylenediaminetetraacetic acid (EDTA)); complexing agents (for example, caffeine, polyvinylpyrrolidone, β-cyclodextrin, or hydroxypropyl-β-cyclodextrin); fillers; monosaccharides; disaccharides and other carbohydrates (for example, glucose, mannose, or dextrin); proteins (for example, serum albumin, gelatin, or immunoglobulin); coloring agents; flavoring agents and diluting agents; emulsifying agents; hydrophilic polymers (for example, polyvinylpyrrolidone); low molecular-weight polypeptides; salt-forming counter ions (for example, sodium); preservatives (for example, benzalkonium chloride, benzoic acid, salicylic acid, thimerosal, phenethyl alcohol, methylparaben, propylparaben, chlorhexidine, sorbic acid, or hydrogen peroxide); solvents (for example, glycerin, propylene glycol, or polyethylene glycol); sugar alcohols (for example, mannitol or sorbitol); suspending agents; surfactants or wetting agents (for example, Pluronic (R), PEG, sorbitan esters, polysorbates such as Polysorbate 20 and Polysorbate 80, Triton, tromethamine, lecithin, cholesterol, or tyloxapol); stabilizers (for example, sucrose or sorbitol); tonicity enhancing agents (for example, alkali metal halides (preferably sodium chloride or potassium chloride, mannitol, or sorbitol); delivery vehicles; diluents; pharmaceutical excipients; and/or pharmaceutical adjuvants.

The main solvent or carrier in the prophylactic and/or therapeutic agent for pregnancy-induced hypertension of the present invention may essentially be either aqueous or non-aqueous. For example, a suitable solvent or carrier may be distilled water for injection or saline, and other substances commonly comprised in compositions for parenteral administration may be added thereto. Examples of the solvents include neutral buffered saline with mixed serum albumin or saline alone. Other examples may include Tris buffer with a pH of about 7.0 to 8.5, or acetic acid buffer with a pH of about 4.0 to 5.5. Such buffers may further comprise sorbitol or suitable alternatives thereof. Furthermore, the prophylactic and/or therapeutic agent for pregnancy-induced hypertension of the present invention may be formulated as a lyophilized product using a suitable pharmaceutical excipient such as sucrose. The optimal pharmaceutical composition is determined by those skilled in the art based on the intended route of administration, delivery method, and target dosage.

The prophylactic and/or therapeutic agent for pregnancy-induced hypertension of the present invention can be produced by a method known to those skilled in the art, such as mixing the optional ingredient described above to the APA-Fc fusion protein as the essential ingredient.

### [Administration and Dosage]

An effective amount of the prophylactic and/or therapeutic agent for pregnancy-induced hypertension of the present invention when used therapeutically varies depending on the content and the purpose of the prophylaxis and/or the treatment. In other words, the dosage level suitable for the treatment varies in part depending on the molecule to be delivered, the route of administration of the polypeptide, and the size (body weight, body surface area, or organ size) and the condition (age and general health conditions) of a patient. Therefore, in order to achieve optimal therapeutic effects, the dosage setting and the route of administration may be varied. A typical dosage may range from about 0.0001 mg/kg to about 100 mg/kg or more of the APA-Fc fusion protein, depending on the above factors. Preferably, the prophylactic and/or therapeutic agent for pregnancy-induced hypertension of the present invention may be injected or administered intravenously. Depending on a half-life and a clearance rate of the formulation, it may be administered daily, every one to two days, every three to four days, weekly, biweekly, or monthly. The frequency of administration depends on pharmacokinetic parameters of the polypeptide in the formulation used. Typically, administration is continued until the dosage reaches a level that achieves the desired effect. The prophylactic and/or therapeutic agent for pregnancy-induced hypertension of the present invention may be administered as a single dose, multiple doses over a long period (with the same or different concentrations per administration), or by continuous infusion. Further refinement of appropriate dosage levels is routinely conducted. The appropriate dosage may also be confirmed using proper dose-response data.

The specific administration route of the prophylactic and/or therapeutic agent for pregnancy-induced hypertension of the present invention follows known methods and is, for example, an oral route, an injection through an intravenous route, an intraperitoneal route, an intratumoral route, an intracerebral route (an intraparenchymal route), an intraventricular route, an intramuscular route, an intraocular route, an intra-arterial route, an intraportal route, and an intralesional route, as well as an intramedullary route, a subarachnoidal route, an intravesical route, an intracardiac route, a transdermal route, a subcutaneous route, or an intraperitoneal route; and a route of administration using an intranasal means, an enteral means, a topical means, a sublingual means, a transurethral means, an intravaginal means, or a rectal means, or a sustained-release system or an implantable device. Optionally, the agent may be administered via bolus injection, continuous infusion, or by an implantable device. Furthermore, localized administration may be achieved by implanting a membrane, sponge, or other suitable material in which a target molecule is adsorbed or encapsulated. When an implantable device is used, the device can be implanted in any suitable tissue or organ, and the target molecule may be delivered via diffusion, time-release bolus, or continuous administration.

### <APA-Fc Fusion Protein>

The present invention also encompasses an APA-Fc fusion protein in which the aminopeptidase A (APA) is bound to an immunoglobulin Fc region via a peptide linker including at least one amino acid sequence selected from the group consisting of SEQ ID NOs: 4 to 9. In the APA-Fc fusion protein of the present invention, the aminopeptidase A (APA) is preferably bound to an immunoglobulin Fc region via a peptide linker including at least one amino acid sequence selected from the group consisting of SEQ ID NOs: 4 to 9. The APA-Fc fusion protein of the present invention has physical properties or a molecular weight that prevent it from passing through the placental barrier, exhibits a pharmacological action that mimics the natural physiological function of the placenta and thus has safety for the mother and the fetus with less side effects, and is also long-lastingly stable and capable of exerting a sustained efficacy in the body. Therefore, it is suitably used as an active ingredient for a prophylactic and/or therapeutic agent for hypertensive disorders of pregnancy. Note that the description provided in the section regarding the prophylactic and/or therapeutic agent for hypertensive disorders of pregnancy is equally applicable to the APA-Fc fusion protein of the present invention.

### <Polynucleotide Encoding the APA-Fc Fusion Protein>

The present invention also encompasses a polynucleotide encoding the aforementioned APA-Fc fusion protein. By introducing a polynucleotide encoding the aforementioned APA-Fc fusion protein in which the APA, the peptide linker, and the Fc region are fused into an expression vector and transforming a suitable host cell with this expression vector, the APA-Fc fusion protein can be produced. Note that the description provided in the section regarding the prophylactic and/or therapeutic agent for hypertensive disorders of pregnancy is equally applicable to the APA-Fc fusion protein of the present invention.

### EXAMPLES

The present invention will now be described in detail with reference to the following Examples and test examples. However, the present invention is not limited to these Examples and the like.

### [Test 1] Production of APA-Fc Fusion Protein

Plasmids were constructed by inserting into a pCAGGS vector a base sequence encoding a prepro-trypsin leader peptide (MSALLILALVGAAVA), a human APA starting from Ala72, and the Fc region of human IgG1. Expi293F cells (Life Technologies Japan Ltd.) were cultured in an Expi293 Expression Medium (Life Technologies Japan Ltd.) at a density of 2.5× 10⁶ cells/mL under conditions of 37° C., 8% CO₂, and shaking at 125 rpm. Various plasmids were introduced into the Expi293F cells using an ExpiFectamine 293 Transfection Kit (Life Technologies Japan Ltd.). The cells were cultured for five days to allow secretion of the APA-Fc fusion protein into the cell culture. The cell culture was subjected to centrifugal separation for 10 minutes at 4° C., a centrifugal force of 6,300 g, and the resulting supernatant was filtered through a filtration filter (PVDF Millex-GV filter with a pore size of 0.22 μm, Merck Ltd.), and the filtrate was subjected to affinity column purification.

A MonoSpin ProA column (GL Sciences Inc.) was conditioned by passing 5 mL of binding buffer (150 mM sodium chloride, 50 mM sodium phosphate buffer, pH 7.0) through it. Then, the filtrate was passed through the column so that the APA-Fc fusion protein was retained in the column. After washing the column by passing therethrough 5 mL of washing buffer (1 M sodium chloride, 50 mM sodium phosphate buffer, pH 7.0), 1 mL of elution buffer (100 mM glycine buffer, pH 3.5) was passed through the column five times so that the APA-Fc fusion protein was eluted from the column. The eluate was immediately mixed with 0.1 mL of neutralization buffer (1 M Tris(hydroxymethyl)aminomethane buffer) for neutralization. The neutralized eluate was placed into a dialysis membrane (Spectra/Por, molecular weight cutoff 140,000), and stirred in 1 L of phosphate-buffered saline (PBS) at 4° C. This procedure was repeated three times to exchange the APA-Fc fusion protein solution to the PBS. The protein concentration of the APA-Fc fusion protein solution was measured using a BCA Protein Assay Kit (Life Technologies Japan Ltd.), with bovine serum albumin as the standard solution.

### [Test 2] Measurement of Hydrolytic Activity of Various APA-Fc Fusion Proteins on Substrate

### (1) Hydrolytic Activity on Angiotensin II

APA-Fc fusion proteins at concentrations of 1.5 or 15 μg/mL were incubated at 37° C. with 10 μM angiotensin II (Peptide Institute, Inc.) in a buffer containing 25 mM Tris(hydroxymethyl)aminomethane, 1 mM calcium chloride, and 0.01% bovine serum albumin (pH 7.5). The reaction was quenched by adding cold methanol at twice the volume of the reaction solution. After centrifugation at 21,500 g for 10 minutes at 4° C., water at 4.5 times the volume of the reaction solution was added to the supernatant. The solution was subjected to centrifugal separation again at 21,500 g for 10 minutes. The resulting supernatant was then subjected to measurement by liquid chromatography-triple quadrupole mass spectrometry (LCMS-8040, Shimadzu Corporation). By using a COSMOSIL 3C18-MS-II column (2.5 μm particle size, 2.0 mm I.D.×50 mm; Nacalai Tesque, Inc.) with a mobile phase composed of water containing 0.1% formic acid (solution A) and acetonitrile containing 0.1% formic acid (solution B), the solutions were charged at a flow rate of 0.4 mL/min. The gradient conditions were as follows: the solution B at 5% was maintained initially, increased to 15% from 0.3 min to 2.5 min, and further to 95% from 2.5 min to 3.5 min. The solution B was held at 95% from 3.5 min to 4.0 min, then decreased back to 5% by 4.1 min, and maintained at 5% until 5.5 min. Angiotensin III was detected by the mass spectrometer using a precursor ion as m/z 466.3 and a product ion as 70.1. The results are shown in FIG. 2 and Table 1.

TABLE 1

| | Vₘₐₓ | | |
|---|---|---|---|
| | (nmol/sec/ | Kₘ | K_{cat/Km} × 10³ |
| | mg protein) | (μM) | (/μM · sec) |
| rhAPA- | 6.20 ± | 21.7 ± | 4.89 |
| Fc | 0.61 | 3.5 | |
| rhAPA- | 101 ± | 44.3 ± | 39.2 |
| (G4S)-Fc | 24 | 11.6 | |
| rhAPA- | 19.7 ± | 28.8 ± | 11.6 |
| (G4S)2-Fc | 3.7 | 7.3 | |
| rhAPA- | 10.5 ± | 21.8 ± | 8.25 |
| (G4S)3-Fc | 1.2 | 4.3 | |
| rhAPA- | 94.5 ± | 41.2 ± | 39.4 |
| (EA3K)-Fc | 20.7 | 9.2 | |
| rhAPA- | 4.70 ± | 26.2 ± | 3.07 |
| (EA3K)2-Fc | 1.23 | 10.5 | |
| rhAPA- | 35.0 ± | 44.3 ± | 13.6 |
| (EA3K)3-Fc | 7.2 | 13.8 | |

### (2) Hydrolytic Activity on Glu-MCA

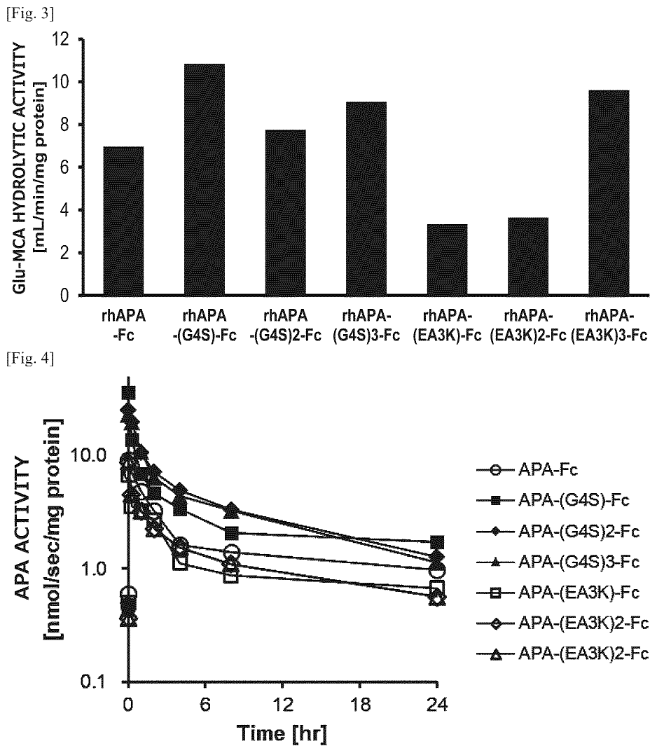

The APA-Fc fusion proteins obtained from the Test 1 were diluted by 10,000-fold after dialysis, and incubated at 37° C. with 50 UM Glu-MCA (Watanabe Chemical Industries, Ltd.), in a buffer containing 25 mM Tris(hydroxymethyl)aminomethane, 1 mM calcium chloride, and 0.01% bovine serum albumin (pH 7.5). The Glu-MCA was a substrate in which the carboxyl group of glutamic acid (Glu) was fluorescently labeled with methylcoumarin amide. The fluorescence (excitation wavelength: 370 nm/fluorescence wavelength: 460 nm) derived from 7-amino-4-methylcoumarin, produced upon hydrolysis of the Glu-MCA, was detected over time using a plate reader (Spark 10M, Tecan Japan). A calibration curve was created based on the fluorescence intensity when 7-amino-4-methylcoumarin (Tokyo Chemical Industry Co., Ltd.) at a known concentration was used as a standard sample, and the amount of 7-amino-4-methylcoumarin generated by hydrolysis was quantified. The results are shown in FIG. 3. In addition, using APA-Fc fusion proteins at known concentrations as standard samples, calibration curves were created based on the AMC generated upon incubation with the Glu-MCA, and concentrations of various APA-Fc fusion proteins in plasma samples were quantified.

As shown in FIG. 2 and Table 1, among the APA-Fc fusion proteins, those employing (G4S), (G4S) 2, (G4S) 3, (EA3K), and (EA3K) 3 as linker exhibited higher hydrolytic activity on angiotensin II as the physiological substrate of APA, as compared to rhAPA-Fc (without linker). On the other hand, as shown in FIG. 3, the hydrolytic activity on another APA substrate, that is the Glu-MCA, was slightly higher in the APA-Fc fusion proteins employing (G4S), (G4S) 2, (G4S) 3, and (EA3K) 3 as linker, as compared to rhAPA-Fc (no linker). It is indicated that the increase in the enzymatic activity as a result of the introduction of the linker depends on the linker sequence and the substrate compound.

### [Test 3] Evaluation of Fetal Transferability of ¹²⁵I-Labeled APA-Fc Fusion Protein in Pregnant Mice

A solution of 83.3 μM APA-Fc fusion protein (rhAPA-Fc) in 50 mM phosphate buffer (pH 7.5) was reacted with 3.7 MBq of Na¹²⁵I and 50 μg/mL of chloramine T at room temperature for two minutes. The reaction was quenched by adding 85 μg/mL of cysteine. The reaction solution was applied to a PD MidiTrap G-25 column (Cytiva), which had been equilibrated with PBS containing 0.1% bovine serum albumin (BSA), and the eluate was collected. The radioactivity of the eluate was measured using a gamma counter (ARC-8001, Japan Raytech Co., Ltd.), and the fraction with high radioactivity (4.98× 10⁵ dpm/pmol) was used for administration to mice. Pregnant ICR mice at day 18 of gestation (Japan SLC, Inc.)

were anesthetized with isoflurane, and 4×10⁶ dpm/kg of [¹²⁵I] rhAPA-Fc dissolved in PBS containing 0.1% BSA was administered via the jugular vein. The fetuses were collected at 1, 5, and 120 minutes after euthanasia of the mice. Ice-cold PBS, in a volume three times the fetal weight, was added to the collected fetuses, followed by homogenization. Bovine serum albumin and trichloroacetic acid were added to reach final concentrations of 0.12% and 15%, respectively. The homogenates were centrifuged at 6,700 g for one minute, and the radioactivity in the resulting precipitate was measured using the gamma counter. The results are shown in Table 2.

TABLE 2

| Time after administration | 1 min | 5 min | 120 min |
|---|---|---|---|
| Fetal radioactivity | 0.0880 ± | 0.151 ± | 0.490 ± |
| (proportion to administered dose) | 0.0602 | 0.053 | 0.057 |

As shown in Table 2, even 120 minutes after the administration of 125T-labeled APA-Fc fusion protein (rhAPA-Fc), 0.5% or less radioactivity of the total dose of administration was detected in the fetuses. This suggests that fetal transferability of rhAPA-Fc in mice is very low.

[Test 4] Evaluation of Blood Stability of Various APA-Fc Fusion Proteins in Mice

Seven-week-old female ICR mice (Japan SLC, Inc.) were anesthetized with isoflurane, and a single intravenous dose of 6.92 mg/kg of various APA-Fc fusion proteins dissolved in PBS containing 0.1% BSA was administered via the jugular vein. Blood was collected from the tail vein, subjected to centrifugation at 6,200 g for five minutes at room temperature, thereby obtaining plasma. The plasma samples were incubated with the Glu-MCA, and fluorescence derived from 7-amino-4-methylcoumarin was detected using a plate reader (see Test 2 above). Pharmacokinetic parameters were calculated based on the plasma concentration profiles of the various APA-Fc fusion proteins using moment analysis. The results are shown in FIG. 4 and Table 3.

TABLE 3

| | AUC_{(0-∞)} | | | | |
|---|---|---|---|---|---|
| | (mg/mL · | CLₜₒₜ | V₀ | V_{dss} | t_{1/2′z} |
| | hr)^{a)} | (mL/hr/kg)^{b)} | (mL/kg)^{c)} | (mL/kg)^{d)} | (h)^{e)} |
| rhAPA- | 34 ± | 37 ± | 153 ± | 674 ± | 17 ± |
| Fc | 4 | 4 | 13 | 315 | 8 |
| rhAPA- | 106 ± | 15 ± | 35 ± | 348 ± | 24 ± |
| (G4S)-Fc | 46 | 9 | 12 | 104 | 15 |
| rhAPA- | 88 ± | 15 ± | 50 ± | 122 ± | 8 ± |
| (G4S)2-Fc | 23 | 4 | 8 | 7 | 1 |
| rhAPA- | 84 ± | 16 ± | 56 ± | 125 ± | 7.8 ± |
| (G4S)3-Fc | 21 | 4 | 9 | 7 | 1.1 |
| rhAPA- | 18 ± | 72 ± | 166 ± | 513 ± | 7.2 ± |
| (EA3K)-Fc | 2 | 10 | 14 | 198 | 3.0 |
| rhAPA- | 30 ± | 41 ± | 95 ± | 320 ± | 8 ± |
| (EA3K)2-Fc | 2 | 2 | 1 | 49 | 1 |
| rhAPA- | 32 ± | 380.4 ± | 108 ± | 308 ± | 7 ± |
| (EA3K)3-Fc | 0 | 0.4 | 13 | 111 | 3 |

a) Plasma concentration-AUC extrapolated to infinite time
b) Systemic clearance
c) Volume of distribution in central compartment
d) Volume of distribution in steady state
e) Terminal half-life

As shown in FIG. 4 and Table 3, among the APA-Fc fusion proteins, those employing (G4S), (G4S) 2, and (G4S) 3 exhibited higher stability in blood, as compared to the rhAPA-Fc (without a linker). On the other hand, those employing (EA3K), and (EA3K) 2, and (EA3K) 3 as linker exhibited similar or higher stability in blood immediately after the administration, but showed lower stability than the rhAPA-Fc eight hours after the administration.

[Test 5] Evaluation of Antihypertensive Effects of Various APA-Fc Fusion Proteins

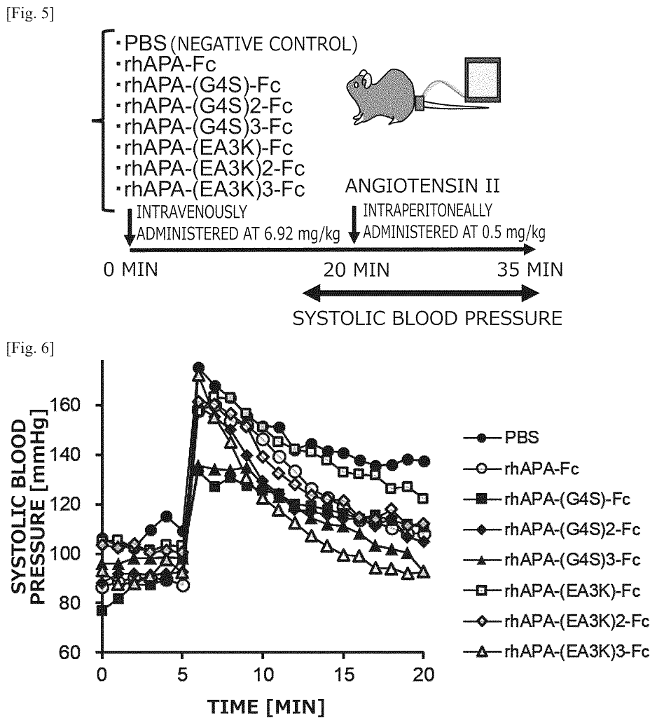

The antihypertensive effect of rhAPA-Fc was evaluated in mice with transient hypertension induced by administration of angiotensin II. Seven-week-old female C57BL/6 mice (Japan SLC, Inc.) were anesthetized by intraperitoneal administration of ketamine (200 mg/kg). Then, either rhAPA-Fc (6.92 mg/kg) or vehicle (PBS containing 0.1% BSA, pH 7.5) was administered intravenously via the jugular vein. Twenty minutes later, angiotensin II (0.5 mg/kg) was administered intraperitoneally. Systolic blood pressure was measured over time using the tail-cuff method with a non-invasive blood pressure monitor (BP-98AL, Softron Co., Ltd.). The test scheme is shown in FIG. 5, and the results are presented in FIG. 6.

As shown in FIG. 6, among the APA-Fc fusion proteins, those employing (G4S), (G4S) 3, and (EA3K) 3 as linkers exhibited superior antihypertensive effects, as compared to rhAPA-Fc (without a linker). In particular, the fusion protein with the (G4S) linker showed a marked and sustained antihypertensive effect immediately after the angiotensin II administration. The fusion protein with the (EA3K) 2 linker showed a gradual decrease in the blood pressure following angiotensin II administration, and the effect was sustained. The fusion protein with the (EA3K) 2 linker exhibited an effect comparable to the rhAPA-Fc (without a linker), whereas the (EA3K) linker version demonstrated little to no antihypertensive effect, similar to PBS (negative control).

### [Test 6] Evaluation of Antihypertensive Effect of APA-Fc Fusion Protein

To evaluate whether the rhAPA-Fc (G4S) 3, which was found to inactivate Ang II in vivo, could ameliorate the pathological state of pregnancy-induced hypertension, its effect on blood pressure, fetal number, and fetal weight was evaluated using a mouse model of pregnancy-induced hypertension. In detail, on the day 10.5 of pregnancy, pregnant ICR female mice (Japan SLC, Inc.) were anesthetized with isoflurane, and the abdominal aorta and vena cava were partially constricted using a plastic tube (inner diameter: 1.2 mm; outer diameter: 2.2 mm) following laparotomy. On the days 15.5 and 16.5 of pregnancy, under isoflurane anesthesia, rhAPA-(G4S) 3-Fc (4 mg/kg) or vehicle (PBS containing 0.1% BSA, pH 7.5) was administered via the tail vein. On the day 17.5 of pregnancy, the systolic blood pressure was measured using the tail-cuff method with a non-invasive blood pressure monitor (BP-98AL, Softron Co., Ltd.), followed by euthanasia and measurement of the number of fetuses and placental weights.

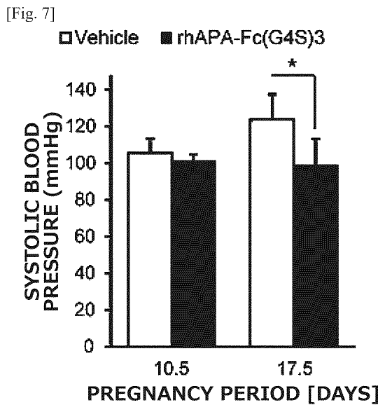

As shown in FIG. 7, in the group administered with vehicle, the systolic blood pressure increased from 105.7±7.6 mmHg on the day 10.5 of pregnancy, immediately before the partial constriction of the abdominal aorta and the vein, to 123.7±13.7 mmHg on the day 17.5 of pregnancy, after the constriction. It indicates successful establishment of the pregnancy-induced hypertension model mice. On the other hand, the group administered with the rhAPA-(G4S) 3-Fc showed no increase in the systolic blood pressure, with values of 101.1±3.7 mmHg before the constriction and values of 99.0±14.2 mmHg after the constriction, which were significantly lower than those of the group administered with the vehicle.

To evaluate the effect of the rhAPA-(G4S) 3-Fc on fetuses in the pregnancy-induced hypertension model mouse, the placental weight, which is considered to be derived from the fetus, was evaluated. The placental weight in the group administered with the vehicle was 123±6 mg, while in the group administered with the rhAPA-(G4S) 3-Fc it was 137±13 mg. It indicates that the administration of the rhAPA-(G4S) 3-Fc significantly increased the placental weight. The fetal loss rate in the pregnancy-induced hypertension model mice was calculated by comparing the number of fetuses at the day 10.5 of pregnancy before the constriction and at the day 17.5 of pregnancy after the constriction. The fetal loss rate was 14.3±21.8% in the vehicle group and 5.9±10.0% in the group administered with the rhAPA-(G4S) 3-Fc. It indicates a tendency for decreased fetal loss rate in the group administered with the rhAPA-(G4S) 3-Fc. These results suggest that the rhAPA-(G4S) 3-Fc can improve fetal development in this pregnancy-induced hypertension model mouse and may exert therapeutic effects on hypertensive disorders of pregnancy in both the mother and the fetus.

The amino acid sequences of SEQ ID NOs: 1 to 12 are as follows.

SEQ ID NO: 1:
MSALLILALVGAAVA
SEQ ID NO: 2:
MSALLILALVGAAVAGTAQDQDICPASEDESGQWKNFRLPDFVNPVHYDLHVKPLLE
EDTYTGTVSISINLSAPTRYLWLHLRETRITRLPELKRPSGDQVQVRRCFEYKKQEYV
VVEAEEELTPSSGDGLYLLTMEFAGWLNGSLVGFYRTTYTENGQVKSIVATDHEPTDA
RKSFPCFDEPNKKATYTISITHPKEYGALSNMPVAKEESVDDKWTRTTFEKSVPMSTY
LVCFAVHQFDSVKRISNSGKPLTIYVQPEQKHTAEYAANITKSVFDYFEEYFAMNYSLP
KLDKIAIPDFGTGAMENWGLITYRETNLLYDPKESASSNQQRVATVVAHELVHQWFG
NIVTMDWWEDLWLNEGFASFFEFLGVNHAETDWQMRDQMLLEDVLPVQEDDSLMS
SHPIIVTVTTPDEITSVFDGISYSKGSSILRMLEDWIKPENFQKGCQMYLEKYQFKNAK
TSDFWAALEEASRLPVKEVMDTWTRQMGYPVLNVNGVKNITQKRFLLDPRANPSQP
PSDLGYTWNIPVKWTEDNITSSVLFNRSEKEGITLNSSNPSGNAFLKINPDHIGFYRVN
YEVATWDSIATALSLNHKTFSSADRASLIDDAFALARAQLLDYKVALNLTKYLKREEN
FLPWQRVISAVTYIISMFEDDKELYPMIEEYFQGQVKPIADSLGWNDAGDHVTKLLRS
SVLGFACKMGDREALNNASSLFEQWLNGTVSLPVNLRLLVYRYGMQNSGNEISWNY
TLEQYQKTSLAQEKEKLLYGLASVKNVTLLSRYLDLLKDTNLIKTQDVFTVIRYISYN
SYGKNMAWNWIQLNWDYLVNRYTLNNRNLGRIVTIAEPFNTELQLWQMESFFAKYP
QAGAGEKPREQVLETVKNNIEWLKQHRNTIREWFFNLLESG
SEQ ID NO: 3:
LINHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGKGT
SEQ ID NO: 4:
GGGGS
SEQ ID NO: 5:
GGGGSGGGGS
SEQ ID NO: 6:
GGGGSGGGGSGGGGS
SEQ ID NO: 7:
EAAAK
SEQ ID NO: 8:
EAAAKEAAAK
SEQ ID NO: 9:
EAAAKEAAAKEAAAK
SEQ ID NO: 10:
MSALLILALVGAAVAGTAQDQDICPASEDESGQWKNFRLPDFVNPVHYDLHVKPLLE
EDTYTGTVSISINLSAPTRYLWLHLRETRITRLPELKRPSGDQVQVRRCFEYKKQEYV
VVEAEEELTPSSGDGLYLLTMEFAGWLNGSLVGFYRTTYTENGQVKSIVATDHEPTDA
RKSFPCFDEPNKKATYTISITHPKEYGALSNMPVAKEESVDDKWTRTTFEKSVPMSTY
LVCFAVHQFDSVKRISNSGKPLTIYVQPEQKHTAEYAANITKSVFDYFEEYFAMNYSLP
KLDKIAIPDFGTGAMENWGLITYRETNLLYDPKESASSNQQRVATVVAHELVHQWFG
NIVTMDWWEDLWLNEGFASFFEFLGVNHAETDWQMRDQMLLEDVLPVQEDDSLMS
SHPIIVTVTTPDEITSVFDGISYSKGSSILRMLEDWIKPENFQKGCQMYLEKYQFKNAK
TSDFWAALEEASRLPVKEVMDTWTRQMGYPVLNVNGVKNITQKRFLLDPRANPSQP
PSDLGYTWNIPVKWTEDNITSSVLFNRSEKEGITLNSSNPSGNAFLKINPDHIGFYRVN
YEVATWDSIATALSLNHKTFSSADRASLIDDAFALARAQLLDYKVALNLTKYLKREEN
FLPWQRVISAVTYIISMFEDDKELYPMIEEYFQGQVKPIADSLGWNDAGDHVTKLLRS
SVLGFACKMGDREALNNASSLFEQWLNGTVSLPVNLRLLVYRYGMQNSGNEISWNY
TLEQYQKTSLAQEKEKLLYGLASVKNVTLLSRYLDLLKDTNLIKTQDVFTVIRYISYN
SYGKNMAWNWIQLNWDYLVNRYTLNNRNLGRIVTIAEPFNTELQLWQMESFFAKYP
QAGAGEKPREQVLETVKNNIEWLKQHRNTIREWFFNLLESGLIGGGGSLINHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGKGTGGGGSHHHH
HHH
SEQ ID NO: 11:
MSALLILALVGAAVAGTAQDQDICPASEDESGQWKNFRLPDFVNPVHYDLHVKPLLE
EDTYTGTVSISINLSAPTRYLWLHLRETRITRLPELKRPSGDQVQVRRCFEYKKQEYV
VVEAEEELTPSSGDGLYLLTMEFAGWLNGSLVGFYRTTYTENGQVKSIVATDHEPTDA
RKSFPCFDEPNKKATYTISITHPKEYGALSNMPVAKEESVDDKWTRTTFEKSVPMSTY
LVCFAVHQFDSVKRISNSGKPLTIYVQPEQKHTAEYAANITKSVFDYFEEYFAMNYSLP
KLDKIAIPDFGTGAMENWGLITYRETNLLYDPKESASSNQQRVATVVAHELVHQWFG
NIVTMDWWEDLWLNEGFASFFEFLGVNHAETDWQMRDQMLLEDVLPVQEDDSLMS
SHPIIVTVTTPDEITSVFDGISYSKGSSILRMLEDWIKPENFQKGCQMYLEKYQFKNAK
TSDFWAALEEASRLPVKEVMDTWTRQMGYPVLNVNGVKNITQKRFLLDPRANPSQP
PSDLGYTWNIPVKWTEDNITSSVLFNRSEKEGITLNSSNPSGNAFLKINPDHIGFYRVN
YEVATWDSIATALSLNHKTFSSADRASLIDDAFALARAQLLDYKVALNLTKYLKREEN
FLPWQRVISAVTYIISMFEDDKELYPMIEEYFQGQVKPIADSLGWNDAGDHVTKLLRS
SVLGFACKMGDREALNNASSLFEQWLNGTVSLPVNLRLLVYRYGMQNSGNEISWNY
TLEQYQKTSLAQEKEKLLYGLASVKNVTLLSRYLDLLKDTNLIKTQDVFTVIRYISYN
SYGKNMAWNWIQLNWDYLVNRYTLNNRNLGRIVTIAEPFNTELQLWQMESFFAKYP
QAGAGEKPREQVLETVKNNIEWLKQHRNTIREWFFNLLESGLIGGGGSGGGGSGGG
GSLINHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGKG
TGGGGSHHHHHHH
SEQ ID NO: 12:
MSALLILALVGAAVAGTAQDQDICPASEDESGQWKNFRLPDFVNPVHYDLHVKPLLE
EDTYTGTVSISINLSAPTRYLWLHLRETRITRLPELKRPSGDQVQVRRCFEYKKQEYV
VVEAEEELTPSSGDGLYLLTMEFAGWLNGSLVGFYRTTYTENGQVKSIVATDHEPTDA
RKSFPCFDEPNKKATYTISITHPKEYGALSNMPVAKEESVDDKWTRTTFEKSVPMSTY
LVCFAVHQFDSVKRISNSGKPLTIYVQPEQKHTAEYAANITKSVFDYFEEYFAMNYSLP
KLDKIAIPDFGTGAMENWGLITYRETNLLYDPKESASSNQQRVATVVAHELVHQWFG
NIVTMDWWEDLWLNEGFASFFEFLGVNHAETDWQMRDQMLLEDVLPVQEDDSLMS
SHPIIVTVTTPDEITSVFDGISYSKGSSILRMLEDWIKPENFQKGCQMYLEKYQFKNAK
TSDFWAALEEASRLPVKEVMDTWTRQMGYPVLNVNGVKNITQKRFLLDPRANPSQP
PSDLGYTWNIPVKWTEDNITSSVLFNRSEKEGITLNSSNPSGNAFLKINPDHIGFYRVN
YEVATWDSIATALSLNHKTFSSADRASLIDDAFALARAQLLDYKVALNLTKYLKREEN
FLPWQRVISAVTYIISMFEDDKELYPMIEEYFQGQVKPIADSLGWNDAGDHVTKLLRS
SVLGFACKMGDREALNNASSLFEQWLNGTVSLPVNLRLLVYRYGMQNSGNEISWNY
TLEQYQKTSLAQEKEKLLYGLASVKNVTLLSRYLDLLKDTNLIKTQDVFTVIRYISYN
SYGKNMAWNWIQLNWDYLVNRYTLNNRNLGRIVTIAEPFNTELQLWQMESFFAKYP
QAGAGEKPREQVLETVKNNIEWLKQHRNTIREWFFNLLESGLIEAAAKEAAAKEAA
AKLINHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGKG
TGGGGSHHHHHHH

### INDUSTRIAL APPLICABILITY

The prophylactic and/or therapeutic agent for hypertensive disorders of pregnancy according to the present invention includes, as an active ingredient, a fusion protein (APA-Fc fusion protein) in which an aminopeptidase A (APA) is bound to the immunoglobulin Fc region via a peptide linker. Thus, the prophylactic and/or therapeutic agent exhibits a pharmacological action that mimics the natural physiological function of the placenta, and has high safety for the mother. Furthermore, since the APA-Fc fusion protein has physical properties and a molecular weight that prevent it from passing through the placental barrier, it also has excellent safety for the fetus. In addition, since the APA-Fc fusion protein has a structure in which the APA is bound to the immunoglobulin Fc region via the specific peptide linker, it has an excellent pharmacological action to degrade angiotensin II to lower the blood pressure, and is also capable of exerting a sustained efficacy in the body. Therefore, the APA-Fc fusion protein can be suitably used as a prophylactic and/or therapeutic agent for hypertensive disorders of pregnancy.

1. A prophylactic and/or therapeutic agent for pregnancy-induced hypertension, comprising an aminopeptidase A (APA) as an active ingredient,
wherein the APA is bound to an immunoglobulin Fc region via a peptide linker.

2. The prophylactic and/or therapeutic agent for pregnancy-induced hypertension according to claim 1,
wherein the peptide linker consists of 5 to 60 amino acids.

3. The prophylactic and/or therapeutic agent for pregnancy-induced hypertension according to claim 1,
wherein the peptide linker comprises at least one amino acid sequence selected from the group consisting of SEQ ID NOs: 4 to 9.

4. The prophylactic and/or therapeutic agent for pregnancy-induced hypertension according to claim 3, further comprising a pharmaceutically acceptable carrier.

5. An aminopeptidase A (APA)-Fc fusion protein, wherein the aminopeptidase A (APA) binds to an immunoglobulin Fc region via a peptide linker comprising at least one amino acid sequence selected from the group consisting of SEQ ID NOs: 10 to 12.

6. The APA-Fc fusion protein according to claim 5, comprising the amino acid sequence of SEQ ID NO: 11.

7. The prophylactic and/or therapeutic agent for pregnancy-induced hypertension according to claim 2,
wherein the peptide linker comprises at least one amino acid sequence selected from the group consisting of SEQ ID NOs: 4 to 9.

8. The prophylactic and/or therapeutic agent for pregnancy-induced hypertension according to claim 7, further comprising a pharmaceutically acceptable carrier.

9. A method of treating pregnancy-induced hypertension in a subject, comprising administering to the subject an effective amount of an aminopeptidase A (APA)-Fc fusion protein comprising an amino acid sequence selected from the group consisting of SEQ ID NOs: 10 to 12.


SEQUENCE LISTING (12 sequences)

SEQ ID NO: 1 - protein, 15 aa, Homo sapiens
MSALLILALVGAAVA

SEQ ID NO: 2 - protein, 903 aa, Homo sapiens
MSALLILALVGAAVAGTAQDQDICPASEDESGQWKNFRLPDFVNPVHYDLHVKPLLEEDTYTGTVSISINLSAPTRYLWLHLRETRITRLPELKRPSGDQVQVRRCFEYKKQEYVVVEAEEELTPSSGDGLYLLTMEFAGWLNGSLVGFYRTTYTENGQVKSIVATDHEPTDARKSFPCFDEPNKKATYTISITHPKEYGALSNMPVAKEESVDDKWTRTTFEKSVPMSTYLVCFAVHQFDSVKRISNSGKPLTIYVQPEQKHTAEYAANITKSVFDYFEEYFAMNYSLPKLDKIAIPDFGTGAMENWGLITYRETNLLYDPKESASSNQQRVATVVAHELVHQWFGNIVTMDWWEDLWLNEGFASFFEFLGVNHAETDWQMRDQMLLEDVLPVQEDDSLMSSHPIIVTVTTPDEITSVFDGISYSKGSSILRMLEDWIKPENFQKGCQMYLEKYQFKNAKTSDFWAALEEASRLPVKEVMDTWTRQMGYPVLNVNGVKNITQKRFLLDPRANPSQPPSDLGYTWNIPVKWTEDNITSSVLFNRSEKEGITLNSSNPSGNAFLKINPDHIGFYRVNYEVATWDSIATALSLNHKTFSSADRASLIDDAFALARAQLLDYKVALNLTKYLKREENFLPWQRVISAVTYIISMFEDDKELYPMIEEYFQGQVKPIADSLGWNDAGDHVTKLLRSSVLGFACKMGDREALNNASSLFEQWLNGTVSLPVNLRLLVYRYGMQNSGNEISWNYTLEQYQKTSLAQEKEKLLYGLASVKNVTLLSRYLDLLKDTNLIKTQDVFTVIRYISYNSYGKNMAWNWIQLNWDYLVNRYTLNNRNLGRIVTIAEPFNTELQLWQMESFFAKYPQAGAGEKPREQVLETVKNNIEWLKQHRNTIREWFFNLLESG

SEQ ID NO: 3 - protein, 229 aa, Homo sapiens
LINHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGT

SEQ ID NO: 4 - protein, 5 aa, synthetic construct
GGGGS

SEQ ID NO: 5 - protein, 10 aa, synthetic construct
GGGGSGGGGS

SEQ ID NO: 6 - protein, 15 aa, synthetic construct
GGGGSGGGGSGGGGS

SEQ ID NO: 7 - protein, 5 aa, synthetic construct
EAAAK

SEQ ID NO: 8 - protein, 10 aa, synthetic construct
EAAAKEAAAK

SEQ ID NO: 9 - protein, 15 aa, synthetic construct
EAAAKEAAAKEAAAK

SEQ ID NO: 10 - protein, 1151 aa, Homo sapiens
MSALLILALVGAAVAGTAQDQDICPASEDESGQWKNFRLPDFVNPVHYDLHVKPLLEEDTYTGTVSISINLSAPTRYLWLHLRETRITRLPELKRPSGDQVQVRRCFEYKKQEYVVVEAEEELTPSSGDGLYLLTMEFAGWLNGSLVGFYRTTYTENGQVKSIVATDHEPTDARKSFPCFDEPNKKATYTISITHPKEYGALSNMPVAKEESVDDKWTRTTFEKSVPMSTYLVCFAVHQFDSVKRISNSGKPLTIYVQPEQKHTAEYAANITKSVFDYFEEYFAMNYSLPKLDKIAIPDFGTGAMENWGLITYRETNLLYDPKESASSNQQRVATVVAHELVHQWFGNIVTMDWWEDLWLNEGFASFFEFLGVNHAETDWQMRDQMLLEDVLPVQEDDSLMSSHPIIVTVTTPDEITSVFDGISYSKGSSILRMLEDWIKPENFQKGCQMYLEKYQFKNAKTSDFWAALEEASRLPVKEVMDTWTRQMGYPVLNVNGVKNITQKRFLLDPRANPSQPPSDLGYTWNIPVKWTEDNITSSVLFNRSEKEGITLNSSNPSGNAFLKINPDHIGFYRVNYEVATWDSIATALSLNHKTFSSADRASLIDDAFALARAQLLDYKVALNLTKYLKREENFLPWQRVISAVTYIISMFEDDKELYPMIEEYFQGQVKPIADSLGWNDAGDHVTKLLRSSVLGFACKMGDREALNNASSLFEQWLNGTVSLPVNLRLLVYRYGMQNSGNEISWNYTLEQYQKTSLAQEKEKLLYGLASVKNVTLLSRYLDLLKDTNLIKTQDVFTVIRYISYNSYGKNMAWNWIQLNWDYLVNRYTLNNRNLGRIVTIAEPFNTELQLWQMESFFAKYPQAGAGEKPREQVLETVKNNIEWLKQHRNTIREWFFNLLESGLIGGGGSLINHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGTGGGGSHHHHHHH

SEQ ID NO: 11 - protein, 1161 aa, Homo sapiens
MSALLILALVGAAVAGTAQDQDICPASEDESGQWKNFRLPDFVNPVHYDLHVKPLLEEDTYTGTVSISINLSAPTRYLWLHLRETRITRLPELKRPSGDQVQVRRCFEYKKQEYVVVEAEEELTPSSGDGLYLLTMEFAGWLNGSLVGFYRTTYTENGQVKSIVATDHEPTDARKSFPCFDEPNKKATYTISITHPKEYGALSNMPVAKEESVDDKWTRTTFEKSVPMSTYLVCFAVHQFDSVKRISNSGKPLTIYVQPEQKHTAEYAANITKSVFDYFEEYFAMNYSLPKLDKIAIPDFGTGAMENWGLITYRETNLLYDPKESASSNQQRVATVVAHELVHQWFGNIVTMDWWEDLWLNEGFASFFEFLGVNHAETDWQMRDQMLLEDVLPVQEDDSLMSSHPIIVTVTTPDEITSVFDGISYSKGSSILRMLEDWIKPENFQKGCQMYLEKYQFKNAKTSDFWAALEEASRLPVKEVMDTWTRQMGYPVLNVNGVKNITQKRFLLDPRANPSQPPSDLGYTWNIPVKWTEDNITSSVLFNRSEKEGITLNSSNPSGNAFLKINPDHIGFYRVNYEVATWDSIATALSLNHKTFSSADRASLIDDAFALARAQLLDYKVALNLTKYLKREENFLPWQRVISAVTYIISMFEDDKELYPMIEEYFQGQVKPIADSLGWNDAGDHVTKLLRSSVLGFACKMGDREALNNASSLFEQWLNGTVSLPVNLRLLVYRYGMQNSGNEISWNYTLEQYQKTSLAQEKEKLLYGLASVKNVTLLSRYLDLLKDTNLIKTQDVFTVIRYISYNSYGKNMAWNWIQLNWDYLVNRYTLNNRNLGRIVTIAEPFNTELQLWQMESFFAKYPQAGAGEKPREQVLETVKNNIEWLKQHRNTIREWFFNLLESGLIGGGGSGGGGSGGGGSLINHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGTGGGGSHHHHHHH

SEQ ID NO: 12 - protein, 1161 aa, Homo sapiens
MSALLILALVGAAVAGTAQDQDICPASEDESGQWKNFRLPDFVNPVHYDLHVKPLLEEDTYTGTVSISINLSAPTRYLWLHLRETRITRLPELKRPSGDQVQVRRCFEYKKQEYVVVEAEEELTPSSGDGLYLLTMEFAGWLNGSLVGFYRTTYTENGQVKSIVATDHEPTDARKSFPCFDEPNKKATYTISITHPKEYGALSNMPVAKEESVDDKWTRTTFEKSVPMSTYLVCFAVHQFDSVKRISNSGKPLTIYVQPEQKHTAEYAANITKSVFDYFEEYFAMNYSLPKLDKIAIPDFGTGAMENWGLITYRETNLLYDPKESASSNQQRVATVVAHELVHQWFGNIVTMDWWEDLWLNEGFASFFEFLGVNHAETDWQMRDQMLLEDVLPVQEDDSLMSSHPIIVTVTTPDEITSVFDGISYSKGSSILRMLEDWIKPENFQKGCQMYLEKYQFKNAKTSDFWAALEEASRLPVKEVMDTWTRQMGYPVLNVNGVKNITQKRFLLDPRANPSQPPSDLGYTWNIPVKWTEDNITSSVLFNRSEKEGITLNSSNPSGNAFLKINPDHIGFYRVNYEVATWDSIATALSLNHKTFSSADRASLIDDAFALARAQLLDYKVALNLTKYLKREENFLPWQRVISAVTYIISMFEDDKELYPMIEEYFQGQVKPIADSLGWNDAGDHVTKLLRSSVLGFACKMGDREALNNASSLFEQWLNGTVSLPVNLRLLVYRYGMQNSGNEISWNYTLEQYQKTSLAQEKEKLLYGLASVKNVTLLSRYLDLLKDTNLIKTQDVFTVIRYISYNSYGKNMAWNWIQLNWDYLVNRYTLNNRNLGRIVTIAEPFNTELQLWQMESFFAKYPQAGAGEKPREQVLETVKNNIEWLKQHRNTIREWFFNLLESGLIEAAAKEAAAKEAAAKLINHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGTGGGGSHHHHHHH